(19) United States
(12) Patent Application Publication
(10) Pub. No.: US 2020/0051257 A1
(43) Pub. Date: Feb. 13, 2020

(54) SCAN ALIGNMENT BASED ON PATIENT-BASED SURFACE IN MEDICAL DIAGNOSTIC ULTRASOUND IMAGING

(71) Applicant: Siemens Medical Solutions USA, Inc., Malvern, PA (US)

(72) Inventors: Frank Sauer, Princeton, NJ (US); Shelby Scott Brunke, Sammamish, WA (US); Andrzej Milkowski, Issaquah, WA (US); Ali Kamen, Skillman, NJ (US); Ankur Kapoor, Plainsboro, NJ (US); Mamadou Diallo, Plainsboro, NJ (US); Terrence Chen, Princeton, NJ (US); Klaus J. Kirchberg, Plainsboro, NJ (US); Vivek Kumar Singh, Princeton, NJ (US); Dorin Comaniciu, Princeton Junction, NJ (US)

(21) Appl. No.: 16/058,067

(22) Filed: Aug. 8, 2018

(51) Int. Cl. G06T 7/37; A61B 8/08; A61B 8/00

(52) U.S. Cl. CPC G06T 7/37; A61B 8/483; A61B 8/4245; A61B 8/085; G06T 2207/20048; A61B 8/5261; G06T 2207/10028; G06T 2207/10136; G06T 2207/30096; A61B 8/5253

(57) ABSTRACT

Imaging from sequential scans is aligned based on patient information. A three-dimensional distribution of a patient-related object or objects, such as an outer surface of the patient or an organ in the patient, is stored with any results (e.g., images and/or measurements). Rather than the entire scan volume, the three-dimensional distributions from the different scans are used to align between the scans. The alignment allows diagnostically useful comparison between the scans, such as guiding an imaging technician to more rapidly determine the location of a same lesion for size comparison.

### BACKGROUND

The present embodiments relate to medical diagnostic imaging. Medical images are stored in various coordinate systems, often relative to the scanner or some arbitrary point in space. This makes it difficult to align and compare scans taken at different times, scanners, modalities and patients, e.g. to relate follow-up studies of the same patient or to quantitatively compare scans across patient populations. For example, ultrasound images representing planar slices of a patient are stacked using freehand ultrasound scanning to provide a three-dimensional (3D) representation of the patient. The pose of the transducer or image plane then allows assembling the individual two-dimensional (2D) ultrasound slices into a 3D volume. Understanding the image information in a 3D spatial context may be highly desirable. When such scanning is repeated for the same patient at a later examination, the pose information for the later scan is not related to the pose of the pervious scan. The coordinate systems from the different scans need to be aligned to allow comparison of the images and/or information from the images. In a thyroid examination example, a sonographer may spend an hour trying to identify corresponding lesions in the later examination that were previously located for the earlier examination.

Alignment of medical images is often done manually or by visual comparison next to each other. Manual alignment may not be accurate and may vary widely between users, making the images less diagnostically reliable. Depending on the use case, there are image-based registration methods to automatically align scans (e.g., registration of follow-up scans to prior scans of the same patient, or registration of scans for different modalities (e.g. CT and PET imaging)). Image-based registration may be computationally expensive and requires large storage to store the entire three-dimensional scan for later registration.

### SUMMARY

By way of introduction, the preferred embodiments described below include methods, computer-readable media, and systems for aligning scans from different times with a medical imager. Imaging from sequential scans is aligned based on patient information. A three-dimensional distribution of a patient-related object or objects, such as an outer surface of the patient or an organ in the patient, is stored with any results (e.g., images and/or measurements). Rather than the entire scan volume, the three-dimensional distributions from the different scans are used to align between the scans. The alignment allows diagnostically useful comparison between the scans, such as guiding an imaging technician to more rapidly determine the location of a same lesion for size comparison.

In a first aspect, a method is provided for aligning scans from different times with a medical imager. A patient is scanned at a first time. The scanning resulting in first scan data representing the patient at the first time. The medical imager scans the patient at a second time. The scanning results in second scan data representing the patient at the second time. The second time is for a different imaging session than the first time. A first surface in three-dimensions is generated and represents the patient at the first time. A second surface in three-dimensions is generated and represents the patient at the second time. A spatial transformation between the first surface and the second surface is determined. First information from the first scan data is compared with second information from the second scan data based on the spatial transformation. An image of the first and second information is displayed.

In a second aspect, a method is provided for aligning scans from different times with a medical ultrasound imager. The medical ultrasound imager three-dimensionally scans with a free-hand transducer a volume of a patient during a first appointment. A three-dimensional distribution represented by scan data from the three-dimensionally scanning during the first appointment and one or more lesions represented by the scan data are determined. A two-dimensional image for the one or more lesions, the three-dimensional distribution, and a location or locations for the one or more lesions are stored. The volume of the patient is three-dimensionally scanned during a second appointment different than the first appointment. The three-dimensional distribution is registered with results from the scanning of the volume during the second appointment. Imaging from the scanning during the second appointment is guided by the registering to be for the one or more lesions.

In a third aspect, a method is provided for aligning scans from different times with a medical imager. A patient is scanned during a first period. A three-dimensional outside surface of the patient during the scanning of the first period is determined, e.g. with a camera that includes a depth sensor. The patient is scanned during a second period at least a day apart from the first period. A three-dimensional outside surface of the patient during the scanning of the second period is determined. The three-dimensional outside surface of the patient from the first period is registered with the three-dimensional outside surface of the patient from the second period. An image from the scanning the patient during the second period is generated based on the registering.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of an embodiment of a method for aligning scans from different times with a medical imager;

FIG. 2 shows an example alignment of coordinates from scans at different times where the alignment is based on patient-specific models;

FIG. 3 is a block diagram of one embodiment of a medical imager system for aligning scans from different times.

### DETAILED DESCRIPTION OF THE DRAWINGS AND SPECIFIC EMBODIMENTS

A patient coordinate system aligns medical scans across scanners and/or time. A shape model of the patient is stored along with medical images and scanner information, such as storing the shape model in the same Dicom file. The shape model relates the scan to the human anatomy instead of some arbitrary (scanner) coordinate system. The transformation between two scans is computed by bringing the shape models into alignment. The alignment is used for comparison and/or to guide scanning to scan the desired locations (e.g., find previously located lesions for comparison of size and/or shape).

Various shape models may be used. For example, with cameras becoming more prevalent in scanning or operating rooms, the reference shape models are outer surfaces of the patient from the camera. As another example, three-dimensional freehand ultrasound examinations use an organ surface (or part of an organ surface) or three-dimensional distribution of landmarks as the shape model. The shape model-based registration aligns coordinate systems without storage of a full three-dimensional scan. The shape model-based registration may guide ultrasound scanning to scan the same lesions without undue effort, such as requiring a few minutes to scan the same lesions instead of an hour hunting for and confirming a lesion of a subsequent scan as being the same lesion as seen in a previous examination.

Freehand-3D (or 3D freehand) ultrasound makes use of a transducer that captures planar images (or 3D images with a limited field-of-view) and puts them together in a volume with the help of tracking technology (e.g., optical or electromagnetic, possibly supported by images based approaches). The tracking system records the pose (position and orientation) of each single capture in a 3D volume. Often, freehand-3D is referred to simply as freehand, where tracking may not be used. As used herein, freehand includes freehand-3D. Freehand 3D ultrasound bridges the gap between 2D and 3D imaging, making use of an ultrasound transducer that yields 2D images and tracking pose (position and orientation) during the acquisition of a sequence of 2D images. The pose information then allows assembly of the individual 2D ultrasound slices into a 3D volume.

In one embodiment, the medical ultrasound imager three-dimensionally scans a volume of a patient during a first appointment according to the freehand-3D paradigm. A transducer acquiring 2D images is tracked with a suitable tracking technology (e.g. with a commercial optical or electromagnetic tracking system). The tracking system tracks the pose (position and orientation) of the transducer so that the individual 2D ultrasound images can be assembled in a 3D coordinate system, resulting in a spatially correct 3D representation of the scanned subject. A three-dimensional distribution derived from the scan data from the three-dimensionally scanning during the first appointment and one or more lesions represented by the scan data are determined. A two-dimensional image for the one or more lesions, the three-dimensional distribution, and a location or locations for the one or more lesions are stored. The volume of the patient is three-dimensionally scanned during a second appointment different than the first appointment. The three-dimensional distribution is registered with results from the scanning of the volume during the second appointment. Imaging of lesions after the 3D scanning during the second appointment is guided by the relating the pose of the current ultrasound image relative to the previously recorded location(s) of the one or more lesions.

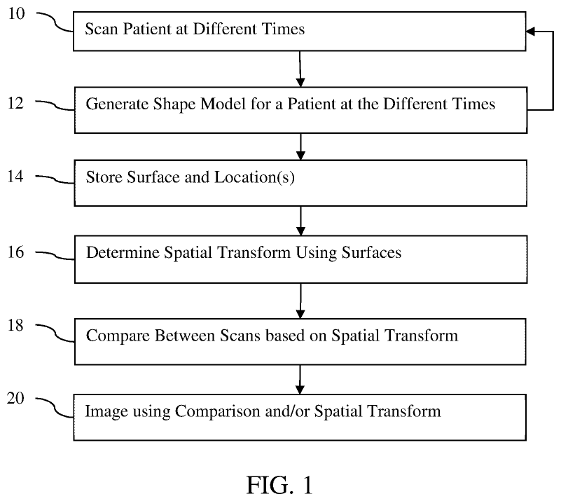

FIG. 1 shows one embodiment of a method for aligning scans from different times with a medical imager. A patient coordinate system is used to align. A three-dimensional distribution of an object or objects of the patient is determined for each scan. The distribution is less than the full 3D volume or scan, such as being a surface. The distribution is stored with the results from the examination and later used to align with the distribution for a subsequent examination. The alignment allows for comparison of information from the different scans.

The method is implemented by a medical diagnostic imaging system, a review station, a workstation, a computer, a PACS station, a server, combinations thereof, or another device for medical imaging. A given scanner performs one or multiple scans of a patient. Different scanners of the same or different modalities (e.g., ultrasound, computed tomography (CT), magnetic resonance (MR), x-ray, positron emission tomography (PET), or single photon emission computed tomography (SPECT)) may perform the scans. A same scanner may perform the scan. A medical imager aligns the results of the scans based on patient-specific shape models. The medical imager performing the generating, storing, determining, comparing, and/or imaging may be one of the medical scanners or a separate server, review station, workstation, or computer. In yet other embodiments, a computer, server, or workstation obtains scan data from memory and a medical scanner is not provided.

The patient shape model alignment may be used in any modality of imaging or across modalities (e.g., align ultrasound with CT or MR). In one embodiment, the alignment provides guidance to scan locations of interest. This guidance may be useful for freehand ultrasound.

The method is implemented in the order shown or a different order. For example, act 14 occurs in between repetition of acts 10-12.

Additional, different, or fewer acts may be performed. For example, act 20 is optional. As another example, act 10 is not performed, but instead scan data is acquired from memory. In yet another example, acts 10-12 are not repeated such as where acts 10-12 are performed for a subsequent examination, and the patient shape model from a previous examination is loaded from memory. In another example, act 18 is not performed.

In act 10, a medical imager scans a patient at a first time. Any modality of medical imager may be used, such as CT, MR, x-ray, ultrasound, PET, or SPECT. The patient is positioned relative to the medical imager and/or the medical imager is positioned relative to the patient for scanning. Any type of scan may be used.

For scanning with an ultrasound scanner, an ultrasound transducer is positioned with acoustic contact to a patient. For scanning a volume of the patient, a volume scanning transducer (e.g., 2D array or multiple 1D array for MPR) or a 2D imaging transducer (e.g., 1D array) may be used. In freehand-3D scanning, a 1D array is translated while planes are imaged. Using a tracking system with a pose sensor, such as a magnetic position sensor, optical sensor, and/or transducer-based scan data, the position of the array and corresponding scan planes are determined, allowing assembly into a volume. The user may manually position the transducer, such as using a handheld probe or manipulating steering wires. Alternatively, a robotic or mechanical mechanism positions the transducer.

For scanning with other modalities, the user is positioned in a bore or by a sensor, source, and/or detector. Emissions from the patient and/or transmissions through the patient are detected.

The volume region of the patient is scanned. Alternatively or additionally, a 2D or just a plane of the patient is scanned. Any organ, such as the thyroid, or parts of a patient may be scanned. One or more objects, such as the heart, an organ, a vessel, fluid chamber, muscle, and/or tissue are within the region.

One or more sets of scan data are obtained. The scan data corresponds to a displayed image (e.g., detected and scan converted data), detector or sensor measurement data, detected data, scan converted data, and/or image processed (e.g., filtered) data. The scan data represents a region of a patient. Data for multiple planar slices may represent the volume region. Alternatively, a volume scan is used.

The scan data is acquired at a given time. The time may correspond to an appointment, examination or imaging session, a period during which continuous scanning occurs (e.g., over 1-20 minutes), or other time relative to scanning by the given modality. During an appointment or imaging session, the patient is scanned once or multiple times. The scanning is to acquire diagnostic information. The imaging session may extend over time within a given day. Other imaging sessions may occur in the same day or be on different days. For example, one appointment is in the morning. After the patient leaves the appointment or scanning room, the patient returns at a different time for another imaging session or appointment.

In one example embodiment, a patient is scanned with ultrasound to locate any lesions in the thyroid. Other organs may be scanned. The lesion is any anatomical abnormality, such as a cyst, scar tissue, void, or tumor.

A volume scan may be performed with freehand or freehand-3D scanning. With the tracked ultrasound transducer, a sequence of images and corresponding pose information are acquired to establish a 3D image volume. The whole thyroid may be acquired with three scans, one for the left side, one for the right side, and one for the isthmus. The volume scan is used to provide context for the location of lesions, so may be sparse or may be a non-sparse or full volume scan. When a lesion is located, one or more 2D images of the lesion may be acquired.

Any ultrasound modality may be used, such as B-mode, color Doppler, elasticity (e.g., acoustic radiation force impulse), and/or other mode. As the ultrasound transducer is tracked, the different images are automatically correlated as to their location and hence as to which lesion is being imaged. In the follow-up scenario, the different images depicting the same lesion may be displayed side-by-side. The images may be cropped so that they only show the lesions with some preset margins.

A careful slow scan for the initial image sequence is performed to obtain a densely sampled volume. The lesions may then be already well imaged in this volume for review and diagnosis based on the volume scan. In another approach, the thyroid is imaged with 3D freehand scanning to establish its dimensions. There is no need to be extra slow and careful with the sweep. In a second step, the user looks for lesions and carefully records representative 2D image or images for the lesions found. One or more images give local insight, such as a single image through the largest cross section, orthogonal images, and/or a dense volume of the lesion.

In act 12, a sensor and an image processor generate a patient-specific shape model. The sensor and image processor generate the shape model during the scanning or after the scanning. The shape model is generated from the scan data or data acquired with the patient positioned for the scanning.

The patient specific shape model is a three-dimensional representation of the patient. Three points, a point and a line, or other combination of information representing a three-dimensional spatial distribution is formed. For example, a three-dimensional surface is formed. The shape model is sparse relative to the sampling for the volume or 3D scanning. Rather than being a collection of voxels with any density of sampling, the shape model is a mesh or other representation of spatial distribution making up fewer than all the voxels (e.g., less than ½ the voxels).

In one embodiment, the shape model is an outer surface of the patient. The outer surface may be the skin of the patient. Any extent of the outer surface may be used, such as a front upper torso. Alternatively, the outer surface includes clothing, such as a hospital gown. The shape of the patient while undergoing scanning or as positioned for scanning (e.g., before or after the scanning) is obtained.

The outer surface is determined with one or more depth sensors. For example, a depth camera (e.g., RGBD) is used. Other depth sensors include a 2D camera with or without transmission of structured light, LIDAR or other imaging modality data. The outer surface may be determined from the scan data. The medical scanning may obtain data representing the outer surface of the patient.

Imaging processing is applied to determine the outer surface from the sensor and/or scan data. This image or images (or other sensor data) are used to compute a personalized shape model of the patient's body. For example, a statistical shape model is fit to the sensor data to personalize the shape to the patient. The resulting shape model reflects the body shape of the patient as well as the pose in which the patient was imaged (prone/supine, head first/feet first etc.).

In another embodiment, the outer surface is determined without scan data or other sensor data. For example, a default or statistical shape model is fit to the patient based on one or more patient characteristics. A height, weight, body mass index, and/or age are used to personalize the average human shape model or to select the shape model for the patient. The patient shape model may be later refined if scan or other sensor data becomes available, such as fitting the selected shape model to the data.

In another embodiment, the shape model corresponds to interior information from the patient. For example, an organ surface is segmented from the scan data as the three-dimensional distribution. One or more point or other landmarks distributed in three dimensions may be detected in the scan images and used as well.

In the thyroid example, the boundaries of the thyroid (e.g., surface of part or all the thyroid) are extracted from the scan data. Any segmentation or delineation of structure in scan data is performed with image processing. The segmentation may be completely manual, with the system providing a tool that lets the user draw the boundaries onto the acquired images. Interactive or semi-automatic approaches give hints, allow placement of seeds, and/or at least partially segment with image processing. In other approaches, an automated segmentation is performed. For example, a statistical shape model is fit to the scan data and interpolates over gaps in between the scan data, allowing mores sparse freehand scanning. As another example, a machine-learned network generates the segmentation.

The spatial distribution or shape model personalized to the patient is a mesh, such as a mesh as representation of thyroid surface. A binarized map, such as voxels labeled as part of or not part of the organ or surface, may be used. Relative locations and distances between landmarks may be used. The spatial distribution can also be a point cloud.

The segmentation of the thyroid or other internal organ or landmarks may yield geometric dimensions, such as height, depth, width, and/or volume. Automatic segmentation of the thyroid with or without automatic calculation of the dimensions (e.g. height, width, depth, volume) assists the user and makes the examination occur more quickly.

In addition to segmentation, the scanning is performed to measure and/or describe lesions. For example, the medical imager includes tools that enable the user to perform width and depth measurements of the lesions shown on the images. The user may click on one side of a lesion's boundary and then on the opposite side of the lesion's boundary to measure. If the images of the scan sequence are oriented in a way as to show width and depth of the lesions in the image plane, the height of the lesions may be obtained from a scan orthogonal to the planes of the images in the sequence. The user may acquire extra images in this orthogonal direction, which would now show height and depth of the lesions. Alternately, the 3D scan is used to create a multiplanar reformatting (MPR) view in the desired orientations. The user may create a dense image sequence just around the location of a lesion to capture the lesion with a good 3D image quality. In other embodiments, automated lesion detection is performed, such as with a segmentation or machine-learned network. Automated classification of any detected lesions may be performed.

Any quantity may be calculated. For example, a distance between two end points is calculated. As another example, an area, circumference, volume, or other spatial measure is performed.

Besides measurements on the geometry of the lesion, the user also may input or annotate about other features: appearance (e.g. hypo/hyper echogenic, sharp/diffuse boundaries, . . . ), and/or type of lesion (e.g. nodule, cyst, tumor, . . . ). Automated classification of the appearance and/or type may be provided, such as using a machine-learned network or other image processing.

The location of any detected lesions is determined. Based on the scan parameters, the location of the lesion relative to the spatial distribution segmented from the patient is determined. The location is assigned based on the surface or spatial distribution. A vector location or voxel label with known voxel size is used.

After scanning in act 12 and determining the 3D spatial distribution (e.g., 3D surface) in act 14, the results of the examination are stored in act 14. The results include the spatial distribution and other information. The other information may be a location of the lesion, an image or scan data representing the lesion, measurements (e.g., quantification of the lesion and/or corresponding organ), and/or scan plane position.

Physician notes, annotations, and/or other information may be stored. Additional information may be stored, such as measurements or parameters related to the patient. For example, radiation dosage or other spatially varying clinical information may be per vertex information for mesh vertices or per voxel information for the volumetric mask model. Information about the scanner (model, location, hospital, etc.) may be saved in the same file.

In one embodiment, a 3D surface of the patient (e.g., outer surface or organ surface) is stored with scan data (e.g., one or more 2D images or 3D voxels). For example, the scan data is images representing one or more lesions. The location of the lesions and/or the scan planes of the images are stored.

The stored information may not include an entire 3D scan. Rather than storing the full volume of data acquired during the examination, the surface or other spatial distribution, the locations, and 2D image or images are stored. The current best estimate of the patient shape model and pose, based on the available data, is stored along with the image data. The patient shape model may be saved as a triangular mesh or as a volumetric binary mask (possibly compressed for efficiency). Storing a patient mesh may also be less of a privacy concern than storing camera images directly.

The location of the lesion and/or scan plane position are indicated with respect to the spatial distribution. The spatial distribution, location of the lesion, and scan plane position are in a same coordinate system. The absolute position of each may be stored. Alternatively, the relative location (e.g., a vector from a defined point) of the location of the lesion and/or location of the scan plane to the spatial distribution is stored. Multiple such instances of different lesions and/or scan planes may have corresponding locations stored.

In alternative embodiments, the scan data (e.g., 2D image or images and/or 3D voxels) are stored without lesion information or even the spatial distribution. The spatial distribution may be extracted from the stored scan data as needed, such as when the scan data is to be registered to scan data from another time. In yet other alternatives, the measurements for an organ or lesion and locations of the lesion or organ are stored with the spatial distribution without storing scan data. The measurements of a lesion over time are to be compared, so the spatial distribution and location of the lesion or organ are sufficient for later comparison.

The information is stored in a memory, such as a database. The information may be stored in a computerized medical record for the patient or hospital treating the patient. In one embodiment, the information is stored in a DICOM file under a custom patient model tag. The medical imager may write the information collected in the examination to a digital storage device, either to the hard drive of the medical imager or to a separate database or archive.

The information being stored is for a given imaging session and/or appointment. The results from the examination are stored for later review and/or comparison. The results may be updated, altered, or created after the examination but before a next examination. For example, scan data is stored during the examination. After the examination ends, the sonographer or physician uses the stored scan data to locate one or more lesions. The lesion information is stored after physician review. The spatial distribution may be created and stored during the examination, during the physician review, or at another time. When created, the spatial distribution is stored. Alternatively, the spatial distribution is not stored, but may be created as needed.

In the thyroid examination example, the user's task is to measure and record the size of the thyroid and to identify and record abnormal anatomy (i.e., lesions) in the thyroid. The medical imager enables the user to record locations of lesions identified on the images. For example, the user clicks on the location in a 2D image where a lesion is seen, and the medical imager records the coordinates. With the coordinates in the 2D image known, and the position and orientation of the image known, the 3D coordinates of the lesion are known. Automated localization of the lesion in the 2D images may be used.

The information created during the examination, including 3D information, is saved. Where the spatial distribution (e.g., surface of the thyroid) is saved, 3D information just for any located regions is saved. In one embodiment, only relevant images (e.g. those showing the views through the center of the lesions or other abnormalities) together with their position information, and the surface of the thyroid with its position information are stored. This reduces the amount of data stored as compared to storing the whole image sequence or whole volume compounded from this image sequence. The images contain the relevant clinical information, and the surface of the thyroid represents the spatial reference for future follow-up scans. A further reduction is possible by cropping the images so that only the image regions that contain a lesion plus a certain margin are stored. Cropping may be done manually or automatically based on a segmentation of the lesion and preset margins. The crop box may automatically be determined from the lesion location, the lesion dimensions, and the preset margins.

A reviewer or radiologist has easy access to the relevant information. The information saved at the end of an examination may be loaded, reviewed and edited off-line. For example, a sonographer performs the examination, and a radiologist later reviews the exam and makes edits (adds notes, findings, . . . ). In a later follow-up examination, the system enables the user to load the previously saved information including the edits.

The scanning of act 10 is repeated. The repetition is for a later examination (e.g., later appointment) for the patient. Hours, days, weeks, months, or years later, the patient is scanned again. For example, at least a day separates the scans, such as for a follow-up examination to compare lesions of the thyroid of the patient now with the lesions for the patient during the previous scan. Alternatively, the later examination is part of a same appointment or hospitalization, such as scanning with a different modality. The repetition of the scanning is performed at a different time and/or imaging session.

The scanning is by a same or different medical imager. The same or different settings may be used. The same or overlapping volume of the patient is scanned.

The generation of the three-dimensional distribution (e.g., outer or organ surface of the shape model) is repeated in act 12. The repetition bases the three-dimensional distribution on the patient during the subsequent examination. For example, the surface of the thyroid is segmented from the scan data resulting from the scanning during the subsequent examination. As another example, an outside surface of the patient is captured by one or more depth sensors with the patient positioned for the scanning of the subsequent examination.

The three-dimensional distribution may be generated during the examination, prior to the examination, or later. For the earlier and/or subsequent examination, the three-dimensional distribution may be generated at any time based on information for the patient for the respective examination.

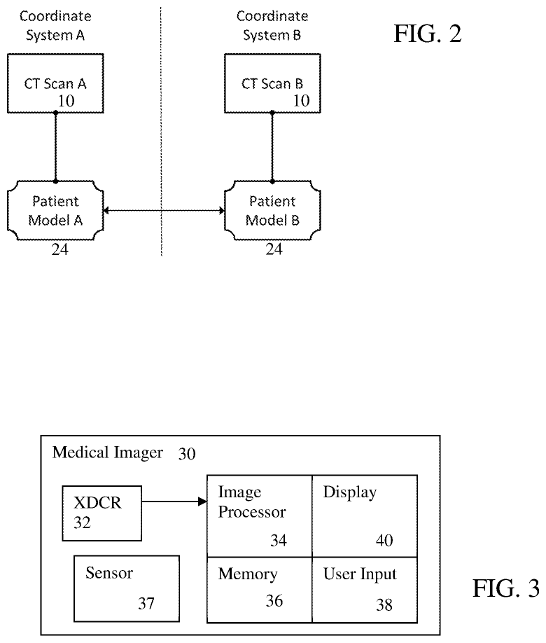

In act 16, the medical imager, such as with an image processor, determines a spatial transformation between the three-dimensional spatial distributions. Rather than storing full 3D scans and registering scan data, the coordinates and/or spatial locations are aligned between scans using the three-dimensional spatial distributions. For example, a patient outside surface or organ surface from one time or period is registered with the patient outside surface or organ surface from another time or period. The patient-specific shape models from different times are registered or aligned to determine the spatial transform of the scanning from the different times. The spatial transform indicates the spatial relationship between the coordinate systems or locations from the scans at different times. As represented in FIG. 2, two scans 10 are brought into alignment by registering their respective patient shape models 24 to one another.

The medical imager enables the user to load the information saved from an earlier examination, including the three-dimensional spatial distribution other than the 3D scan data. The saved information is loaded for registration with information for a current or other examination.

Information in addition to the shape models or three-dimensional distribution may be used to align. For example, fidelity information about the estimated patient model (e.g., radiation dose by location) may be leveraged to achieve more accurate and robust registration. Registration may be further improved by adding approximate anatomical information of other parts of the body, such as including internal scan data information for registration with the outside surfaces or vise versa.

The registration is rigid or non-rigid. For example, if the shape models are stored as structured surface meshes (vertices+faces), rigid alignment may be performed by minimizing the mean square distance between the vertices. Non-rigid alignment uses a deformation model that reflects the real deformation between human shapes and poses.

In the thyroid example, the medical imager spatially registers a new volume to the old volume using the thyroid surface. The thyroid of the previous examination is registered with the thyroid of the current follow-up examination. Different solutions are possible, such as a surface-to-surface registration with an iterative closest point algorithm. For example, automatic registration of previous thyroid to current thyroid is provided as mesh-to-mesh registration with the iterative closest point algorithm. The registration of follow-up scan to previous scan may be based on volumes and/or surfaces: volume to volume, volume to surface, and/or surface to surface. For some applications, landmark based registration may be used.

In act 18, the medical imager compares information from the earlier scan data with information from the subsequent scan data. The comparison is based on the spatial transformation. The spatial transform aligns locations from one examination with locations for another examination.

In one embodiment, the comparison is of images. An image from one examination may be displayed adjacent to an image from another examination. The images are positioned for display based on the alignment. Alternatively, one image is subtracted from the other image, and the resulting difference is displayed. The images are aligned prior to subtraction so that the difference represents a change due to treatment or time, such as a change in size of a lesion. In another alternative, an image is overlaid on another image after alignment. For example, one image maps to color and the other image maps to intensity. An image is generated from both the color and the intensity.

In another embodiment, the information compared is of lesions. The alignment is used so that lesions from the same locations in the patient are compared (i.e., the same lesion is identified in both examinations). The medical imager, using the spatial transform, enables the user to compare the lesions from both examinations. The comparison may be of images of the lesion. The location of a scan plane from the earlier examination and the spatial transformation are used to orient a scan or imaging plan in a current examination. An image in the current examination representing the lesion from the same perspective is generated. The images or quantities extracted from the images may be compared.

Lesion characteristics may be compared. The spatial transformation is used to identify the same lesion. The size, shape, or other characteristic of the lesion from different times may be compared, such as in a graph, as a difference, as a ratio, or in another representation. Descriptions (e.g., annotations) may be compared, such as differences in appearance. The medical imager allows population and storage of reports that contain measurements from the follow-up examination together with measurements from the previous scan, and information on the changes between previous examination and follow-up examination.

In the thyroid example, a location from the scan data of one time is compared to a scan plane position of the transducer array during the scanning of another time. The user or medical imager selects a best image (e.g., largest lesion cross-section of a lesion) that shows a previously found lesion in the follow-up scan. Based on the registration, the medical imager picks the image in the follow-up image sequence that is closest to the location of the lesion. The medical imager may determine the size of the lesion's cross-section (auto-segmentation) in a set of neighbor images. The image where the lesion has the largest cross-section is chosen as the best image (or best representative image) of the lesion for the current examination.

In another example, the standard thyroid report includes the three dimensions of the lesions: height, width, and depth. For each lesion, two images are acquired with orthogonal orientations through the lesion center (one showing width and depth, one height and depth). The spatial transformation is used to locate the same lesion and/or to provide for the same scan planes relative to the same lesion. Alternately, the user performs a local sweep (i.e., 3D freehand scan) and acquires a dense set of images covering the lesion. From this 3D information, the measurement of the dimensions is derived, such as by automatic segmentation. From this dense set of images, best or representative images are extracted (e.g., largest lesion cross-section) for display and storage. The spatial transformation is used to identify the correct lesion or location of the lesion for segmentation, scanning, and/or measurement. The user may also save the local 3D volume for later reference. To reduce the amount of stored data, the 3D volume may be cropped around the lesion.

Other information may be compared. The comparison may be by aligned or overlaid images to compare qualitatively. The comparison may be of measurements or quantities to compare quantitatively. The comparison may be both qualitative and quantitative.

In act 20, a display device displays an image. The medical image is mapped from intensities (scan data) representing tissue and/or other objects in the patient. The scanning provides intensities. For example, the intensities are B-mode or flow mode values from ultrasound scanning. As another example, the intensities are generated by beamforming prior to detection. After detection, the scalar values for the intensities may be scan converted, providing intensities in a different format. Scalar values from any point along a CT or MR imaging pipeline may be used. By mapping scalar values to a dynamic range and with an image gain, display values are generated. The medical image is a color or a gray-scale image.

In one embodiment, the medical image is a volume rendered image of a volume of tissue scanned by ultrasound, such as a 3D rendered image of the thyroid. Using surface rendering, projection, path tracing, or other volume rendering technique, the data representing the volume is rendered to a 2D image. An image processor (e.g., a graphics processing unit) renders the image on the display. In other embodiments, the image is a 2D image from scalar values representing a plane or interpolated to a plane from voxel values. The medical image is generated from intensities representing just a plane. A plane is scanned, and the image is generated from the scan. Alternatively, a plane is defined in a volume. The intensities from volume or 3D scanning representing the plane are used to generate the medical image, such as with MPR. Interpolation may be used to determine the intensities on just the plane from the volume data set.

The image includes information from the different scans. The comparison and/or spatial transform are used for generating the image. For example, the image includes quantities from the same lesion, determined based on the spatial transform and/or comparison. The quantities from the different times are displayed as a graph, table, chart, or other representation. As another example, the image includes representations of the patient as scanned at the different times. The representations are aligned in pose and/or size based on the spatial transform. The representations may be overlaid on each other in the image or displayed side by side. The image may include information from both scans combined into a single image, such as a subtraction. The spatial transform allows for the subtraction or overlay by aligning the locations from the different scans prior to subtraction.

The imaging occurs after or as part of the ongoing scanning of the patient. For example, the image is generated during the subsequent scanning. The imaging uses the registration between the shape models, such as providing a quantitative or qualitative comparison. The pose, size, or spatial arrangement of the image may be based on the registration. A difference as reflected by a mathematical difference, side-by-side display, or other difference is determined based on the registration and included in the image.

The information displayed may be entirely from the subsequent or initial scanning. The information may be from both the subsequent and initial scanning.

The medical imager enables the user to save information created during the examination, including 3D information, for later imaging. More than one previous scan may be saved. This enables differences over time, such as charting the growth of a lesion over several examinations. In this case, the latest of the previous examinations may be used to locate the lesions (via registration) in the current examination where the locations of the lesions in the other examinations are previously determined and are used to provide comparative measurements. All relevant earlier measurements may be combined by averaging, variance calculation, or a collection. The previous measurements are included as part of the information created and stored for each new examination. Only the latest dataset needs to be loaded when performing the next follow-up examination as the latest includes the whole history of examinations. Alternatively, separate storage for the separate examinations is used.

In one embodiment for 3D-freehand ultrasound, the information from the different scans is used to guide the imaging of the current scan. During a current (e.g., subsequent) appointment, the scanning is guided based on the spatial transformation and the known locations of the previously recorded lesions. The registration is used to indicate a placement of an imaging plane relative to the location or locations for the one or more lesions. The locations of lesions in the newly acquired volume or images are predicted based on the spatial transformation and the locations from a previous scan. The locations in the patient that correspond to the locations marked in the old volume may be found and identified in the current or new examination.

For example, the locations of lesions in a thyroid are recorded in the previous examination. The user's task is to image those same lesions and locations in the follow-up scan for comparison. If the image sequence recorded for the follow-up scan samples the thyroid densely enough, this is a matter of identifying the matching images in the current sequence to the locations. The 3D coordinates of a lesion location from the previous scan are used to finds the closest 2D image in the follow-up image sequence based on the spatial transformation. The closest 2D image of the aligned coordinate systems may be calculated as a point-to-plane or other distance. The 2D image from the current examination is displayed on the monitor. A marker may show the expected lesion location. As the estimated location may not be completely accurate, the system enables the user to scroll through the neighbor images in the sequence, identify the one that best shows the lesion (e.g. central view with largest diameter), and mark the image.

If the initial image sequence of the current scan is sparse, the user may be guided to perform a 2D scan at the lesion locations. The medical imager guides the user to find the right locations with the transducer. Since (a) the transducer pose is tracked, (b) the coordinates of the current and past scans are registered, and (c) the coordinates of the lesions are known, the medical imager displays an indication to guide the user where to position the scan plane. For example, a semi-transparent model of the thyroid with markers at the lesion locations and the current location of the imaging plane are displayed. To see the transducer location with respect to the target location helps the user to understand in which direction to move the transducer to approach and scan the lesion.

To move the transducer to find a target lesion with known coordinates, optical feedback may be used. The optical feedback on the monitor shows the spatial relationship of target and transducer image plane. Other optical feedback may be used. For example, the target and/or the transducer depiction change colors (e.g. from red=far away to yellow=getting close to green=on target). A separate traffic light showing those colors may be used. Arrows may point to the nearest lesion. The image may also indicate which of the lesions to be imaged have already been imaged, such as a table with check marks or via color coding of the lesion locations.

Alternatively or additionally, audio feedback is used. For example, a beep sounds when the transducer's imaging plane includes the target coordinate. As another example, the audio signal gets louder or more frequent as the transducer is getting closer to the target. The audio signal may turn off or change after the target has been reached, preventing undesired audio when the user explores the neighborhood around the target to find the best view of the target lesion. The audio is enabled again when the transducer is moved away a certain distance from the target.

The guidance provides information from both scans. The locations from the previous scan are indicated relative to the current scan optically and/or audibly. The spatial transform from the shape models indicates the relationship between the two scans. The position and/or image of the current scan are also indicated. This feedback based on the alignment allows a user to quickly find previously imaged lesions, making the subsequent examination occur more rapidly and with greater reliability than a manual search.

In one embodiment, the examination is made more efficient by guiding the user in the workflow. The user is prompted to record the initial image sequence with a sweep, such as a freehand sweep to establish the thyroid space. Then, the medical imager asks the user to determine the dimensions of the thyroid or thyroid part from the image sequence. The user may be asked to confirm the results of an automated segmentation. The user is then guided through the different steps/phases of the examination in a structured way to image lesions, such as lesions identified in other examinations.

In addition to searching for previously identified lesions from other appointments, the user may search for any new or additional lesions. A manual or automated search may be performed. The alignment is used to rule out previously found lesions for identifying new lesions.

FIG. 3 shows a medical diagnostic imager system 30 for aligning scans from different times. The system 30 is a medical diagnostic ultrasound, CT, MR, PET, SPECT, x-ray or other scanner. In other embodiments, the image system 30 is a computer, workstation, database, server, or other imaging system for generating images from scan data. In the example embodiment below, the system 30 is an ultrasound system.

The system 30 implements the method of FIG. 1, the method of FIG. 2, or a different method. The system 30 aligns scans from different times based on a spatial distribution, such as one or more surfaces of the patient. Rather than storing entire 3D scans, one or more 2D images, lesion information, and a reference spatial distribution are stored and used for alignment, comparison, and/or imaging in subsequent scans.

The system 30 includes an image processor 34, a memory 36, a display 40, a transducer 32, sensor 37, and a user input 38. Additional, different, or fewer components may be provided. For example, the system 30 includes a transmit beamformer, receive beamformer, B-mode detector, Doppler detector, harmonic response detector, contrast agent detector, scan converter, filter, combinations thereof, or other now known or later developed medical diagnostic ultrasound system components. As another example, the system 30 does not include the transducer 32, such as where the system 30 is a CT or MR imaging system. In yet another example, the sensor 37 is not provided, such as where the surface is determined from scan data. As another example, a tracking system for determining pose of the transducer 32 is provided, such as for freehand-3D.

The transducer 32 is a piezoelectric or capacitive device operable to convert between acoustic and electrical energy. The transducer 32 is an array of elements, such as a one-dimensional, multi-dimensional, or two-dimensional array. The transducer 32 may include a position or pose sensor and is used for freehand 3D scanning. Alternatively, the transducer 32 is a wobbler for mechanical scanning in one dimension and electrical scanning in another dimension.

The system 30 uses the transducer 32 to scan a volume and/or a plane. Electrical and/or mechanical steering allows transmission and reception along different scan lines. Any scan pattern may be used. Ultrasound data representing a plane or a volume is provided in response to the scanning. The ultrasound data is beamformed by a beamformer, detected by a detector, and/or scan converted by a scan converter. The ultrasound data may be in any format, such as polar or Cartesian coordinates, Cartesian coordinate with polar coordinate spacing between planes, or another format. In other embodiments, the ultrasound data is acquired by transfer, such as from a removable media or over a network. Other types of medical data representing a volume may also be acquired.

The sensor 37 is a depth camera, depth sensor, projector and camera, or another sensor for generating a surface of the patient. The sensor 37 is positioned in a calibrated or fixed location relative to the imager system 30 or detector for medical imaging, so that the spatial relationship of the sensor 37 to the scan data is known. The sensor 37 is positioned to capture a surface of the patient as positioned to be or as being scanned by the imager 30.

The memory 36 is a buffer, cache, RAM, removable media, hard drive, magnetic, optical, or other now known or later developed memory. The memory 36 may be a single device or group of two or more devices. The memory 36 is shown within the system 30 but may be outside or remote from other components of the system 30.

The memory 36 stores the scan data, location of scan planes, locations of lesions, a generated surface or other spatial distribution of the patient, lesion measurements, images, and/or other information from one or more scans. For example, the memory 36 stores an outer surface from the sensor 37 and/or an inner organ surface from scan data with the locations of one or more lesions and/or scan planes for one or more lesions. Measurements of an organ and/or lesions may be stored. The information from an examination is stored for later use to align with a current examination for imaging.

For real-time imaging, the scan data bypasses the memory 36, is temporarily stored in the memory 36, or is loaded from the memory 36. Real-time imaging may allow delay of a fraction of seconds, or even seconds, between acquisition of data and imaging. For example, real-time imaging is provided by generating the images substantially simultaneously with the acquisition of the data by scanning. While scanning to acquire a next or subsequent set of data, images are generated for a previous set of data. The imaging occurs during the same imaging session used to acquire the data. The amount of delay between acquisition and imaging for real-time operation may vary. In alternative embodiments, the ultrasound data is stored in the memory 36 from multiple previous imaging sessions and used for imaging without concurrent acquisition.

The memory 36 is additionally or alternatively a computer readable storage medium with processing instructions. The memory 36 stores data representing instructions executable by the programmed image processor 34 for measurement point determination. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The user input device 38 is a button, slider, knob, keyboard, mouse, trackball, touch screen, touch pad, combinations thereof, or other now known or later developed user input devices. The user may operate the user input device 38 to position measurement calipers, segment, or otherwise interact with the medical imager 30.

The image processor 34 is a general processor, digital signal processor, three-dimensional data processor, graphics processing unit, application specific integrated circuit, field programmable gate array, digital circuit, analog circuit, combinations thereof, or other now known or later developed device for processing medical image data. The image processor 34 is a single device, a plurality of devices, or a network. For more than one device, parallel or sequential division of processing may be used. Different devices making up the image processor 34 may perform different functions, such as a same or different processors for generating images, registering surfaces or sparse spatial distribution, volume rendering, and/or guiding scanning. In one embodiment, the image processor 34 is a control processor or other processor of a medical diagnostic imaging system. In another embodiment, the image processor 34 is a processor of an imaging review workstation or PACS system.

The image processor 34 is configured by hardware, firmware, and/or software. For example, the image processor 34 operates pursuant to stored instructions to perform various acts described herein, such as acts 12, 16, 18, and/or 20 of FIG. 1. In one embodiment, the image processor 34 is configured to generate a shape model for each scan or examination, determine a spatial transform between shape models from different scans or examinations, and/or generate images including information from a comparison and/or the spatial transformation.

The image processor 34 may be configured to generate a graphic indicating a point, scan plane position, and/or shape model. For example, a 3D point is represented relative to an organ surface with one or more graphics indicating positioning of a current scan plane. The image processor 34 is configured to calculate a value, such as a measurement of a lesion area, volume, or length. The image processor 34 is configured to generate an image or images, such as generating spatially registered images or an image of measurements over time.

The display device 16 is a CRT, LCD, plasma, monitor, projector, printer, or other now known or later developed display device. The display 40 is configured by loading an image from the processor into a display buffer. Alternatively, the display 40 is configured by reading out from a display buffer or receiving display values for pixels.

The display 40 is configured to display a medical image or images, such as a volume rendering, MPR images, plane graphics, calipers, measurement graphics, and/or user interface tools. Overlaid and/or side-by-side images from different examinations may be displayed simultaneously.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for aligning scans from different times with a medical imager, the method comprising:
scanning a patient at a first time, the scanning resulting in first scan data representing the patient at the first time;
scanning, by the medical imager, the patient at a second time, the scanning resulting in second scan data representing the patient at the second time, the second time being for a different imaging session than the first time;
generating a first surface in three-dimensions, the first surface representing the patient at the first time;
generating a second surface in three-dimensions, the second surface representing the patient at the second time;
determining a spatial transformation between the first surface and the second surface;
comparing first information from the first scan data with second information from the second scan data based on the spatial transformation; and
displaying an image of the first and second information.

2. The method of claim 1 wherein generating the first and second surfaces comprises generating the first and second surfaces as outer surfaces of the patient.

3. The method of claim 2 wherein generating the first and second surfaces comprises generating with a depth camera.

4. The method of claim 2 wherein generating the first and second surfaces comprises generating the outer surfaces from the first and second scan data.

5. The method of claim 2 wherein generating the first and second surfaces comprises generating the outer surfaces from shape models fit to a characteristic of the patient at the first and second times, respectively.

6. The method of claim 1 wherein the medical imager comprises an ultrasound scanner, and wherein scanning the patient at the first and second times comprises freehand-3D scanning with a tracked one-dimensional transducer array, and wherein generating the first and second surfaces comprises generating the first and second surfaces as an organ surface from the first and second scan data, respectively.

7. The method of claim 6 further comprising storing a first location of a first lesion from the first scan, and wherein comparing comprises determining a second location of the first lesion in the second scan using the spatial transformation.

8. The method of claim 6 wherein comparing comprises comparing a lesion characteristic from the first and second times.

9. The method of claim 6 wherein comparing comprises comparing a location from the first scan data to a scan plane position of the transducer array during the scanning of the second time.

10. The method of claim 1 wherein comparing comprises aligning the first scan data with the second scan data based on the spatial transformation.

11. The method of claim 1 wherein the first time is prior to the second time, and further comprising storing the first surface with the first scan data without storing an entire three-dimensional scan.

12. The method of claim 1 wherein determining the spatial transformation comprises registering the first surface with the second surface with rigid or non-rigid alignment.

13. The method of claim 1 further comprising storing an image of a lesion of the patient from the scanning of the first time, the image of the lesion of the cropped to the lesion.

14. The method of claim 1 further comprising guiding during the scanning of the second based on the spatial transform.

15. The method of claim 14 wherein guiding comprises displaying a spatial indication of a lesion.

16. A method for aligning scans from different times with a medical ultrasound imager, the method comprising:
three-dimensionally scanning, by the medical ultrasound imager with a tracked transducer, a volume of a patient during a first appointment;
determining a three-dimensional distribution represented by scan data from the three-dimensionally scanning during the first appointment and one or more lesions represented by the scan data;
storing an image for the one or more lesions, and storing a location or locations for the one or more lesions and the three-dimensional distribution related to a first coordinate system;
three-dimensionally scanning the volume of the patient during a second appointment different than the first appointment, the scanning being in a second coordinate system;
registering the three-dimensional distribution with results from the scanning of the volume during the second appointment, thereby linking the first and second coordinate systems of the first and second appointments; and
imaging of the one or more lesions during the second appointment, the one or more lesions located during the image in the second coordinate system guided by the lesion locations in the first coordinate system.

17. The method of claim 16 wherein determining the three-dimensional distribution comprises locating an organ surface or locating landmarks.

18. The method of claim 16 wherein storing the location or locations comprises storing a plane position relative to the three-dimensional distribution and wherein storing the image comprises storing the image cropped to the one or more lesions.

19. The method of claim 16 wherein registering comprises registering the three-dimensional distribution from the first appointment with another three-dimensional distribution from the scanning of the second appointment.

20. The method of claim 16 wherein imaging comprises indicating placement of an imaging plane relative to the location or locations for the one or more lesions.

21. A method for aligning scans from different times with a medical imager, the method comprising:
scanning a patient during a first period;
determining a three-dimensional outside surface of the patient during the scanning of the first period;
scanning the patient during a second period at least a day apart from the first period;
determining a three-dimensional outside surface of the patient during the scanning of the second period;
registering the three-dimensional outside surface of the patient from the first period with the three-dimensional outside surface of the patient from the second period;
generating an image from the scanning the patient during the second period, the image based on the registering.

22. The method of claim 21 wherein determining during the first and second periods comprises determining with one or more depth sensors.

23. The method of claim 21 wherein generating the image comprises generating the image with a difference of the patient from the scanning from the second period from the scanning form the first period, the difference determined based on the registering.
